(19) United States
(12) Patent Application Publication
(10) Pub. No.: US 2006/0184161 A1
(43) Pub. Date: Aug. 17, 2006

(54) Flexible shaft system having interchangeable end effectors

(75) Inventors: Tracy D. Maahs, Rancho Santa Margarita, CA (US); Richard C. Ewers, Fullerton, CA (US)

Correspondence Address: LEVINE BAGADE HAN LLP, 2483 EAST BAYSHORE ROAD, SUITE 100, PALO ALTO, CA, 94303, US

(73) Assignee: USGI Medical Inc., San Clemente, CA (US)

(21) Appl. No.: 11/060,231

(22) Filed: Feb. 16, 2005

(51) Int. Cl. A61B 18/18; A61B 18/04

(52) U.S. Cl. 606/2; 606/27; 606/33; 606/41

(57) ABSTRACT

Flexible shaft systems having interchangeable end effectors are described herein. A shaft with a flexibility sufficient to allow advancement through a lumen of an endoscopic device has an interface at its distal end for engaging a removable end effector tool. The shaft and end effector tool are configured to enable endoluminal deployment through a patient, e.g., through the esophagus and into the stomach. The removable end effector tool may comprise any number of different tools, such as graspers, forceps, scissors, snares, needles, etc., each being interchangeable upon the flexible shaft distal end. Once a procedure is done within the patient, the flexible shaft may be withdrawn and another distal end effector tool may be interchanged. The new end effector tool and flexible shaft can then be reintroduced into the patient.

### BACKGROUND OF THE INVENTION

### Field of the Invention

The present invention relates to end effector tools interchangeable upon a flexible shaft. In particular, the tools and methods described below relate to various end effector tools which are interchangeable upon a flexible shaft to effect various endoluminal procedures.

Various end effector tools are widely available for medical instruments, particularly for laparoscopic surgery. Such instruments are typically used to access, e.g., the peritoneal cavity of a patient through small incisions in the abdominal wall. Depending upon the desired procedure, the tools having the appropriate tip end are inserted through the incision to perform the surgery. The tools include end effectors configured for various procedures, e.g., grasping tissue via forceps, cutting via scissors or needle knives, etc.

Most such laparoscopic tools have tips which are permanently connected. But because of the high cost of such tools, reuse of instruments, or part of the instruments, other laparoscopic tools having replaceable tips have also been developed. Examples of such instruments having replaceable tips may be seen in further detail in U.S. Pat. Nos. 5,810,879 and 6,595,984 both to Microline, Inc. (Beverly, Mass.), the entireties of which are each incorporated herein by reference.

However, the use of such tools are limited to laparoscopic procedures which nonetheless require incisions through the skin. Moreover, the nature of laparoscopic tools require straight-line access to the treatment site within the body and the risk of perforating neighboring organs and structures. As such, many of these types of devices are typically unsuitable for low-profile endoluminal delivery through the body, e.g., transesophageally.

Moreover, the difficulties inherent in transmitting forces over a non-linear and non-rigid shaft present additional problems unaddressed by laparoscopic tools. This may be due to difficulties in applying, deploying and/or deforming such devices with low-profile end effectors disposed at significant distances from a medical practitioner, i.e., due to an inability to convey adequate force to the devices and/or end effectors along desired vectors across the significant distances. These difficulties may be exacerbated when the end effectors are coupled to the distal ends of flexible shafts.

Additionally, conventional endoluminal tools which are typically advanced and used through the lumen of conventional endoscopes are limited in size and utility. Conventional endoluminal tools are usually advanced through a side port defined in the endoscope handle which provides access to a working lumen. However, the lumen through the entry port is usually bent at an angle relative to the working lumen and because of this bend, only tools of limited size may be advanced through the handle and into the working lumen.

Thus, known laparoscopic instruments, even those with removable tips, as well as conventional endoluminal tools present limitations which fail to address minimally invasive endoluminal procedures.

### BRIEF SUMMARY OF THE INVENTION

In manipulating tissue within a patient's body, an elongate shaft having a tool on or near the distal end of the shaft may be utilized. Such tools may be used in endoluminal procedures where the tools are delivered through an endoscope. However, several different tools may be required for performing a single endoluminal procedure. An example of a tool which may be advanced endoluminally within a patient and having a flexible shaft may be configured to interchange distal end effectors.

Such a tool may have a shaft sufficiently flexible to be advanced through a lumen of an endoscopic device. The distal end of the flexible shaft may have an end effector with a tool, e.g., to grasp and/or manipulate tissue folds within the stomach, removably attached to effect a procedure. Once a procedure has been completed or during a procedure, the flexible shaft may be withdrawn proximally through the lumen while maintaining a position of the endoscopic body within the stomach. Another distal end effector tool may then be interchanged and the new end effector tool and flexible shaft may be reintroduced into the stomach through the second lumen to complete another procedure, if so desired.

Although many different configurations are possible, one variation of interchangeable end effector assembly may generally have the flexible shaft with a handle assembly operably connected to the proximal end of the shaft. The handle assembly may generally comprise any number of handle configurations for providing a grip and actuation mechanism or lever. Moreover, the handle assembly may be detachable from the proximal end of the shaft. The interchangeable end effector may be removably attachable at the distal end of the flexible shaft.

The end effector may be comprised generally of an end effector body which has an end effector tool at its distal end. A tool actuation member may extend proximally from the body for insertion and engagement within a receiving channel of an end effector engagement member protruding distally from the flexible shaft. The tool actuation member may become engaged within an end effector engagement member and manipulated to actuate the end effector tool. When the end effector body is fully engaged and secured onto the distal end of the flexible shaft, the outer surface of the end effector body may lie flush with the outer surface of the flexible shaft so as to present an atraumatic surface to the tissue being manipulated. The coupling described above is intended to be illustrative of an example for mechanically connecting an end effector and flexible shaft and is not intended to be construed as limiting. Other types of mechanical coupling or attachment mechanisms that are known in the art are intended to be included within the scope of this disclosure.

The end effector may be removed from the flexible shaft and replaced with any number of end effector tools. For example, end effector tools such as alligator-type forceps, hook-tip forceps, conventional graspers such as DeBakey-type graspers, conventional scissors such as Metzenbaum-type scissors, snares, expandable graspers, puncture needles, needle knives, etc., may be utilized. These examples illustrating various end effector tools are merely for illustrative purposes only and are not intended in any way to be limiting in scope. Other variations and tools that are practicable are intended to be within the scope of interchangeable end effector tools.

Aside from attachment of interchangeable distal end effector tools on a single flexible shaft, interchangeable end effectors may also be utilized on other endoluminal surgical tools as shown in U.S. patent application Ser. No. 10/734,562 filed Dec. 12, 2003, which is incorporated herein by reference in its entirety. For instance, interchangeable end effectors may be incorporated upon one or more articulatable arms extending from the distal end of a steerable and rigidizable endoscopic device.

Such a device may be advanced through the esophagus into a stomach and articulated to a tissue region of interest. The steerable distal portions of the articulatable arms may be manipulated to bring the end effector tools into proximity to the tissue to effect a procedure, e.g., mucosectomy, tissue approximation, etc. During the procedure or after, the articulatable arms themselves may be withdrawn proximally through main body and one or both of the distal end effectors may be interchanged and reintroduced into the stomach to complete the procedure or effect another procedure.

### BRIEF DESCRIPTION OF THE DRAWINGS

FIG. 1A shows a shape-lockable endoscopic assembly advanced within a stomach of a patient with a tool having an interchangeable end effector on a flexible shaft deployed therethrough.

FIG. 1B illustrates how a full-sized endoluminal tool is prevented from entry into the working lumen of a conventional endoscope handle.

FIGS. 1C and 1D illustrate how an endoluminal tool having an interchangeable end effector may be advanced through the endoscope handle and into the working lumen.

FIG. 2A shows an illustrative side view of one variation of an end effector tool interchangeably removable from the distal end of an elongate flexible shaft.

FIG. 2B shows a variation of the interchangeable end effector tool which may be coupled, electrically and/or fluidly, to a respective power source or fluid source.

FIGS. 3A to 3J illustrate examples of possible end effector tools which may be utilized interchangeably on the distal end of a flexible shaft.

FIGS. 4A and 4B illustrate one method for operably engaging the end effector body to the flexible shaft.

FIG. 5A shows an illustrative view of one variation of an endoscopic device having a rigidizable body and interchangeable distal end effectors positioned upon articulatable arms.

FIG. 5B illustrates advancement of the main body shaft through the esophagus and into the stomach and the articulation of a tissue region of interest.

FIGS. 6A and 6B illustrate the articulatable arms of the device of FIGS. 5A and 5B and the interchanging of one or both of the distal end effectors.

### DETAILED DESCRIPTION OF THE INVENTION

In manipulating tissue within a patient's body, a tissue manipulation tool having a distal tip may be advanced (transorally, transgastrically, etc.) into the patient, e.g., into the stomach. The tissue may be engaged or grasped and any number of procedures may be performed upon the tissue regions of interest. For instance, examples of creating and forming tissue folds within the stomach may be seen in further detail in U.S. patent application Ser. No. 10/735,030 filed Dec. 12, 2003, which is incorporated herein by reference in its entirety.

In manipulating tissue within a patient's body, an elongate shaft having a tool on or near the distal end of the shaft may be utilized. Such tools are generally utilized in endoluminal procedures where the tools are delivered through an endoscope. Generally, several different tools are required for performing a procedure endoluminally. A variation of a tool which may be advanced endoluminally within a patient may generally comprise a flexible shaft having a distal end of the shaft configured to accept interchangeable distal end effectors. Such a device may enable greater flexibility and facilitate various endoluminal procedures.

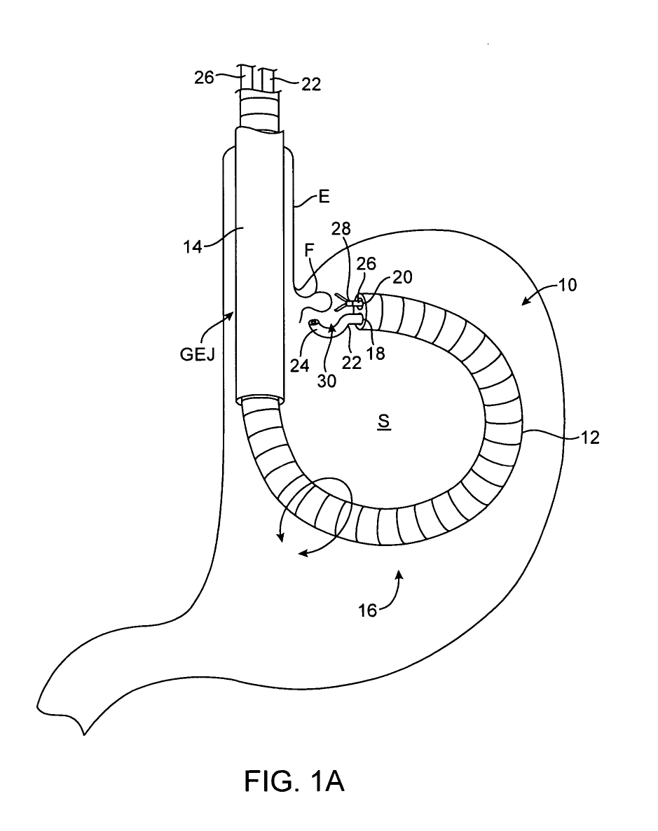

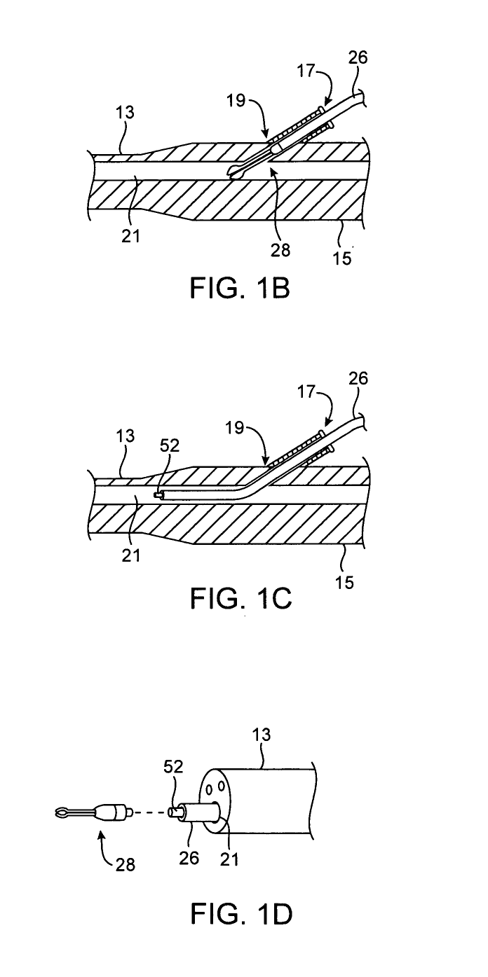

An endoluminal tool having a flexible shaft and interchangeable distal end effectors may be utilized for tissue manipulation procedures within the stomach with any number of endoluminal devices. As illustrated in FIG. 1A, one such example is shown in which a shape-lockable endoscopic assembly 10 may be advanced into a patient's stomach S per-orally and through the esophagus E. Such an endoscopic assembly 10 may generally comprise an endoscopic device which may have a distal portion which may be articulated and steered to position its distal end anywhere within the stomach S. Once desirably configured, assembly 10 may then be locked or rigidized to maintain its shape or configuration to allow for procedures to be performed on the tissue utilizing any number of tools delivered through the assembly 10. Shape-lockable assembly 10 and its variations are described in further detail in U.S. patent application Ser. No. 10/734,562 filed Dec. 12, 2003, which is incorporated herein by reference in its entirety.

Shape-lockable assembly 10 may be generally comprised of shape-lockable endoscopic body 12 having an articulatable distal portion 16. The endoscopic body 12 may define at least first and second lumens 18, 20, respectively, through the endoscopic body 12 through which one or more tools may be deployed into the stomach S. An optional thin wall sheath 14 may be disposed through the patient's mouth, esophagus E, and possibly past the gastroesophageal junction GEJ into the stomach S. Shape-lockable body 12 may be advanced through esophagus E (and through sheath 14, if utilized) and into stomach S while disposed in a flexible state.

Distal steerable portion 16 of endoscopic body 12 may be then articulated to an orientation, e.g., whereby distal portion 16 facilitates engagement of tissue near and/or inferior to the patient's gastroesophageal junction GEJ. Accordingly, distal steerable portion 16 may comprise a number of steering features, as described in further detail in U.S. patent application Ser. No. 10/734,562, incorporated above. With distal steerable portion 16 disposed in a desired configuration or orientation, endoscopic body 12 may be reversibly shape-locked to a rigid state such that the endoscopic body 12 maintains its position within the stomach S. Various methods and apparatus for rigidizing endoscopic body 12 along its length are also described in further detail in U.S. patent application Ser. No. 10/734,562, incorporated above.

An endoscope utilized for visualization, e.g., gastroscope 22, having an optionally steerable distal portion 24 may be advanced through first lumen 18 to provide imaging of the tissue region of interest. Flexible shaft 26 may be advanced through second lumen 20 until interchangeable distal end effector 28, shown in this example as graspers, projects from the distal opening of second lumen 20. Interchangeable end effector 28 may be utilized to effect a procedure 30, e.g., to grasp and/or manipulate tissue fold F within stomach S. Once done, flexible shaft 26 may be withdrawn proximally through second lumen 20 while maintaining a position of endoscopic body 12 within stomach S. Another distal end effector tool may then be interchanged with distal end effector 28 and the new end effector tool and flexible shaft 26 may be advanced back into stomach S through second lumen 20 to complete another procedure, if so desired.

Alternatively, flexible shaft 26 and end effector 28 may be introduced within second lumen 20 prior to advancing endoscopic body 12 into a patient. With end effector 28 disposed within lumen 20 proximally of or within distal steerable portion 16, endoscopic body 12 may be advanced through the esophagus E and into stomach S, where distal steerable portion 16 may be articulated into a desirable position. Once articulated, end effector 28 may then be advanced through the remaining portion of second lumen 20 and out of endoscopic body 12 into stomach S.

In yet another alternative, flexible shaft 26 may be maintained within second lumen 20 during intubation and/or extubation. In this case, distal end effector 28 may be configured to have a diameter or profile which is larger than a diameter of second lumen 20. The larger diameter of end effector 28 may prevent the proximal removal of the tool from endoscopic body 12; however, the entire endoscopic body 12 may be removed from a patient with end effector 28 protruding from second lumen 20 and once outside the patient, end effector 28 may then be interchanged or removed and the endoscopic body 12 may be re-introduced into the patient. This procedure may be repeated as many times as necessary or desired.

One example of how a flexible shaft 26 having an interchangeable end effector 28 may be advanced through a conventional endoscope is shown in FIGS. 1B to 1D. FIG. 1B shows a partial cross-sectional representative view of a conventional endoscope handle 15 with an endoscope body 13 extending from the handle 15 and entry port 17 extending at an angle from handle 15. Because entry port 17 extends at an angle, a distal end effector tool 28 with a flexible shaft 26, e.g., for manipulating tissue, has difficulty in negotiating bend 19 where the entry port lumen joins working lumen 21, which extends through endoscope body 13.

Accordingly, as shown in FIG. 1C, a flexible shaft 26 having an interchangeable end effector tool may have its end effector removed to allow shaft 26 to be advanced through entry port 17, past bend 19, and through working lumen 21. Flexible shaft 26 may then be advanced to its distal opening, shown in FIG. 1D, until the distal end of shaft 26 extends from working lumen 21. Once exposed, any number of distal end effector tools, e.g., end effector tool 28 and as described in further detail below, may be connected to flexible shaft 26. End effector tool 28 may then be withdrawn proximally partially into working lumen for advancing the endoscope body 13 into a patient. Alternatively, end effector tool 28 may be positioned to remain extending from working lumen 21 and advanced into the patient.

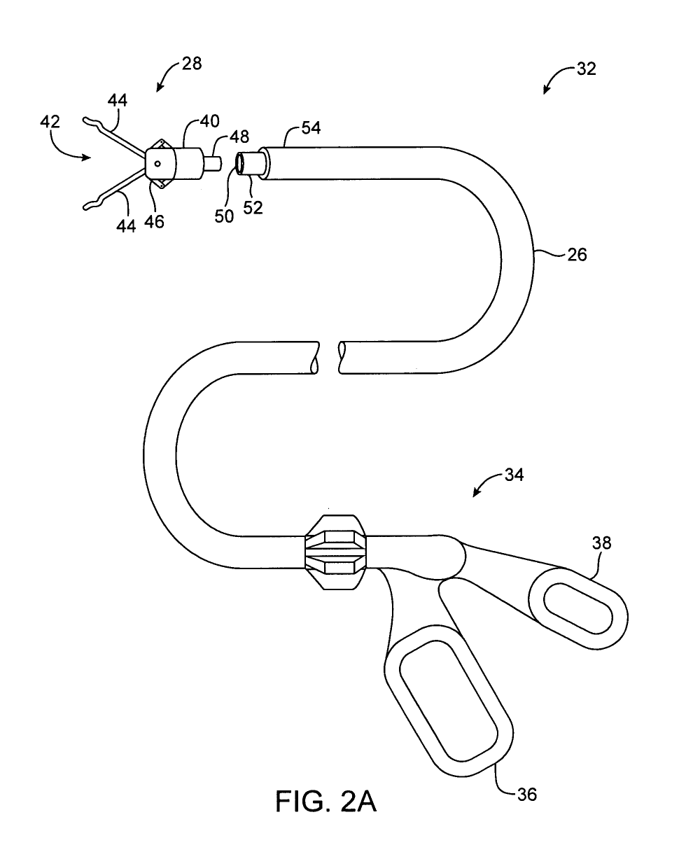

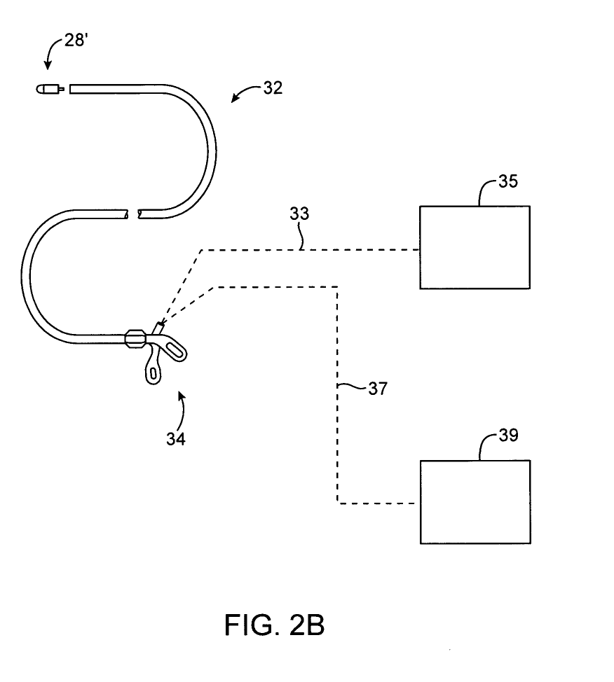

Although many different configurations are possible, one variation of interchangeable end effector assembly 32 is shown in the illustrative side view of FIG. 2A. Assembly 32 may generally have flexible shaft 26 with handle assembly 34 operably connected to the proximal end of shaft 26. Handle assembly 34 may generally comprise any number of handle configurations for providing a grip 36 and actuation mechanism or lever 38 which a practitioner may manipulate. Moreover, handle assembly 34 may be configured to be detachable to the proximal end of shaft 26 through any number of known mechanism connections.

Interchangeable end effector 28 may be removably attachable at the distal end of flexible shaft 26. End effector 28 may be comprised generally of end effector body 40 which has an end effector tool 42 at its distal end. In the illustration of FIG. 2A, end effector tool 42 is shown as having grasper arms, e.g., Babcock-type graspers for manipulating soft tissue, which may be articulated via linkage mechanism 46 housed within body 40. Tool actuation member 48 may extend proximally from body 40 for insertion and engagement within receiving channel 50 of end effector engagement member 52, which may protrude distally from flexible shaft 26. Tool actuation member 48 may become engaged within end effector engagement member 52 and manipulated to actuate end effector tool 42, as described below. When end effector body 40 is fully engaged and secured onto the distal end of flexible shaft 26, the outer surface of body 40 is preferably configured to lie flush with outer surface 54 of flexible shaft 26 so as to present an atraumatic surface to the tissue being manipulated.

FIG. 2B shows a variation of the interchangeable end effector system in which the end effector may be energized or configured for fluid delivery. In this variation, end effector 28′ may be configured to have an atraumatic or blunted end. Handle assembly 34 may be connected to a power source 35 via an electrical connection 33, for instance, such that end effector 28′ is in electrical communication with power source 35. Power source 35 may be configured to generate a number of different energy forms, e.g., RF, microwave, ultrasound, laser energy, etc.

Alternatively, handle assembly 34 may be configured to connect via feedline 37 to a fluid reservoir and/or pumping source 39, for example, to delivery cryogenic fluid or gas through flexible shaft 26 to end effector 28′. Cryogenic fluid or gas may be circulated through end effector 28′ to enable cyro-ablation procedures. Alternatively, others fluids may be dispensed from reservoir 39 through end effector 28′ for other procedures, e.g., saline may be dispensed or fluids for drug delivery through end effector 28′ may be utilized. Although end effector 28′ is shown in FIG. 2B as a blunt atraumatic tip, energy delivery (in its various forms) or fluid delivery, may be incorporated into various different end effector tools, as practicable. Some examples are described below and are intended to be merely illustrative and not limiting in any manner.

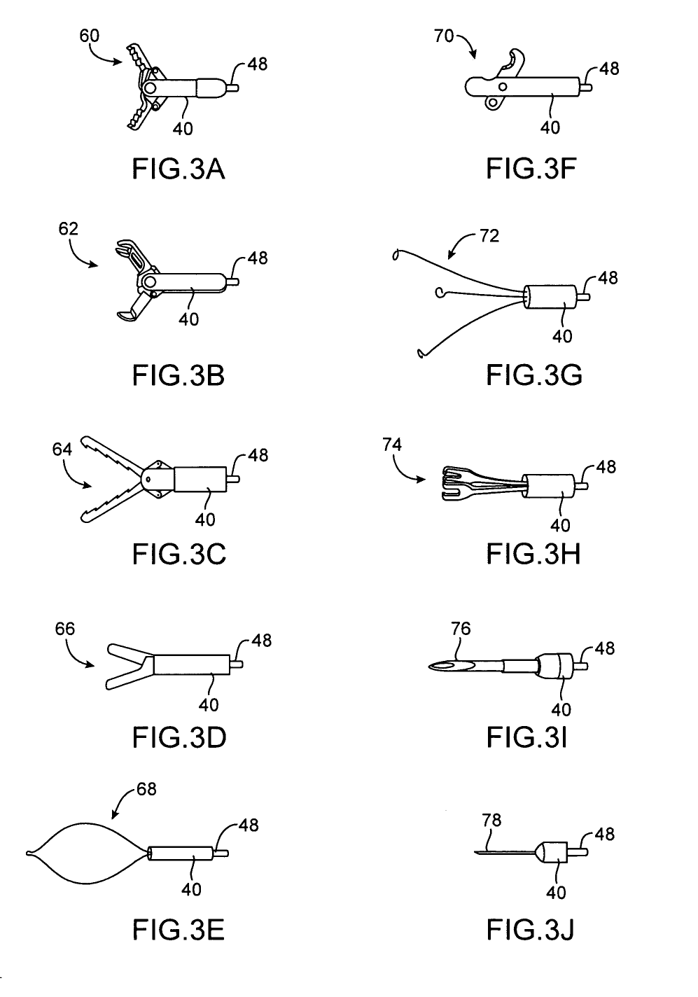

End effector 28 may be removed from flexible shaft 26 and replaced with any number of end effector tools. For instance, FIGS. 3A to 3J illustrate examples of possible end effector tools which may be utilized interchangeably on the distal end of flexible shaft 26. The examples shown are merely illustrative and are not intended to be limiting in any way. Other examples and types of end effector tools not illustrated may be modified to be securable upon the end of flexible shaft 26 and are intended to be within the scope of this disclosure.

FIG. 3A shows one example of alligator-type forceps 60 typically used for manipulating foreign bodies within a patient on end effector body 40, which may be articulated via manipulation of actuation member 48. FIG. 3B shows another example of hook-tip forceps 62 and FIG. 3C shows yet another example of a grasper, e.g., DeBakey-type graspers 64, which may also be energizable with RF energy for tissue ablation procedures. FIGS. 3D and 3F shows examples of end effector having scissors, e.g., Metzenbaum-type scissors 66 in FIG. 3D and flexible scissors 70, for cutting tissue or other structures such as sutures within the patient's body.

Other examples of end effectors may be seen in FIG. 3E, which shows a snare 68, symmetric or asymmetric, which may be utilized to grasp tissue structures or objects within the body. Other graspers may be seen in the examples of FIG. 3G, which shows a three-armed expandable grasper 72, and FIG. 3H, which shows a two-armed pronged grasper. Aside from graspers and snare-type end effectors, other types of tools may be utilized for interchangeable end effector. For instance, FIG. 3I shows a puncture needle 76 which may project from end effector body 40. Needle 76 may be solid or hollow. A hollow needle body 76 may be utilized to carry various anchors or to deliver chemical agents. Another example is shown in FIG. 3J which shows a needle knife 78 projecting from end effector body 40. Needle knife 78 may be energizable to perform tissue ablation and cutting procedures. As mentioned above, these examples illustrating various end effector tools are merely for illustrative purposes only and are not intended in any way to be limiting in scope. Other variations and tools that are practicable are intended to be within the scope of interchangeable end effector tools.

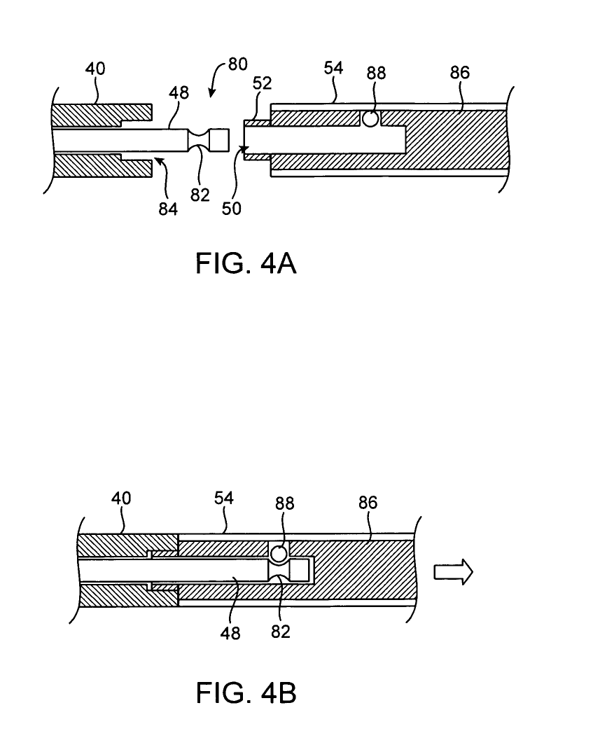

The end effector body may be connected to the distal end of flexible shaft 26 in a variety of ways. One example is shown in FIGS. 4A and 4B, which illustrate one method for operably engaging the end effector body 40 to the flexible shaft 26 such that the end effector tool may be articulated via manipulations of the handle by a user. In this illustration, end effector engagement assembly 80 generally comprises tool actuation member 48 projecting distally from body 40. Actuation member 48 may be inserted within receiving channel 50 defined within the distal end of the flexible shaft. Once inserted as shown in FIG. 4B, groove or notch 82 defined near the proximal end of actuation member 48 may come into engagement with a locking member contained within channel 50. Locking member is illustrated as ball member 88, but it may also include any number of locking members such as springs, screws, collars, etc. As ball member 88 engages groove or notch 82, end effector engagement member 52 may also be received within engagement receiving channel 84 defined within end effector body 40.

Engagement member 52 may be engaged securely within receiving channel 84 in various ways, e.g., engagement member 52 may be threaded for a screw-fastener engagement within receiving channel 84. Alternatively, engagement member 52 may be friction-fitted within channel 84. In another alternative, engagement member 52 may also define a detent or protrusion for interlocking within a complementary notch within channel 84. Once end effector body 40 has been securely engaged to the flexible shaft, actuation member 86, which may be translatable within the flexible shaft relative to outer surface 54, may be actuated via the handle assembly 34 in the direction as shown by the arrow to urge the engaged actuation member 48 proximally (or distally) to effect movement of the end effector tool for carrying out a procedure.

Other examples for engagement of a removable end effector tip to a rigid shaft are shown in further detail in U.S. Pat. Nos. 5,810,879 and 6,595,984 to Microline, Inc. (Beverly, Mass.), which have been incorporated herein above by reference in their entireties. Moreover, various methods may be utilized for transmitting forces over the flexible shaft to the interchangeable end effector. Examples of such methods and apparatus are disclosed in further detail in U.S. patent application Ser. No. 11/035,993 filed Jan. 14, 2005 (Attorney Docket No. 021496-004400US), which is incorporated herein by reference in its entirety.

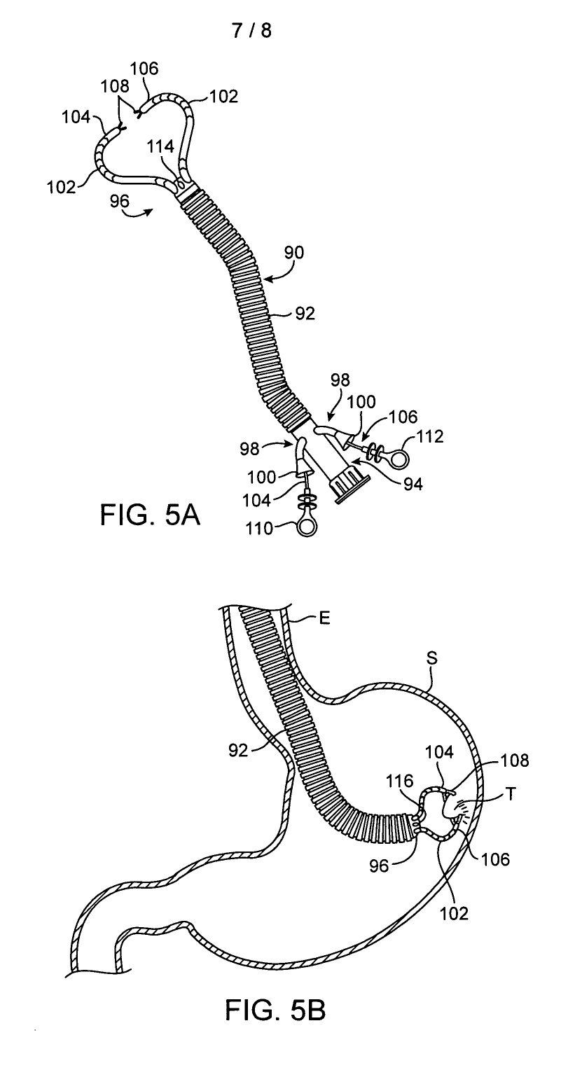

Aside from attachment of interchangeable distal end effector tools on a single flexible shaft, interchangeable end effectors may also be utilized on other endoluminal surgical tools as shown in U.S. patent application Ser. No. 10/734,562 filed Dec. 12, 2003, which has been incorporated herein above by reference in its entirety. In the assembly shown in FIG. 5A, main body 90 in one variation may be comprised generally of main body shaft 92, which may be reversibly shape-locked to a rigid state as described above in Ser. No. 10/734,562 and in U.S. patent application Ser. No. 10/458,060 filed Jun. 9, 2003, which is incorporated herein by reference in its entirety. The proximal end 94 of main body shaft 92 may define one or more articulation channels 98 which define entry ports 100 through which a first and second end effector control 104, 106, respectively, may be coupled to a first and second end effector control handle 110, 112, respectively.

The distal end portion 96 of main body shaft 102 may have one or more articulatable arms extending therefrom and scope lumen 114 defined at the distal end through which an endoscope having visualization capabilities or some other visualization element, such as a CCD imager, may be positioned. One or both of the articulatable arms may have a steerable distal portion 102 for facilitating positioning and manipulation of the tissue region of interest. Moreover, each of the articulatable arms may also have interchangeable first and second end effector bodies 104, 106 each having a number of end effector tools 108 thereon.

FIG. 5B illustrates advancement of main body shaft 92 through esophagus E to stomach S and articulation to a tissue region of interest T while the main body shaft 92 is disposed in a flexible state. An endoscope 116 may be positioned within main body shaft 92 to provide visualization of the tissue region of interest T. Steerable distal portions 102 of the articulatable arms may be manipulated to bring the end effector tools 108 into proximity to the tissue to effect a procedure, e.g., mucosectomy, tissue approximation, etc.

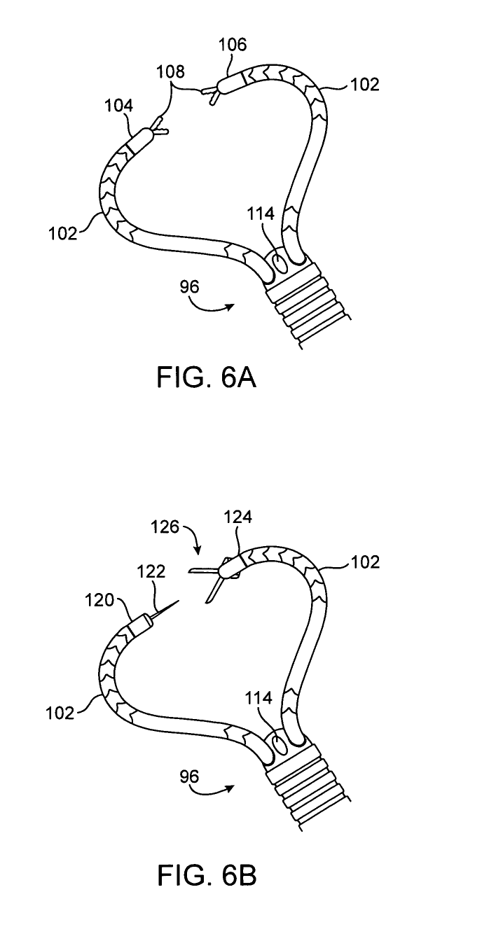

During the procedure or after, main body shaft 92 may be withdrawn from stomach S or the articulatable arms themselves may be withdrawn proximally through main body shaft 92 and one or both of the distal end effectors may be interchanged and reintroduced into stomach S to complete the procedure or effect another procedure. FIG. 6A shows a close-up detail view of the articulatable arms 102 and their respective end effector tips 104, 106. The tools 108 may be interchanged upon the articulatable arms 102, as shown in FIG. 6B, to any other desired end effector tools such as first end effector body 120 having a energizable needle knife 122 and second end effector body 124 having graspers 126 for manipulating soft tissue. These end effectors are merely illustrative and other end effector tools may be utilized, as necessary or desired.

It may be appreciated that the systems, methods, and devices described herein are applicable to diagnostic and surgical procedures in any location within a body, particularly any natural or artificially created body cavity. Such locations may be disposed within the gastrointestinal tract, urology tract, peritoneal cavity, cardiovascular system, respiratory system, trachea, sinus cavity, female reproductive system and spinal canal, to name a few. Access to these locations may be achieved through any body lumen or through solid tissue. Moreover, a variety of procedures may be performed with the systems and devices described above. The following procedures are intended to provide suggestions for use and are by no means considered to limit such usage: laryngoscopy, rhinoscopy, pharyngoscopy, bronchoscopy, sigmoidoscopy, colonoscopy, and esophagogastroduodenoscopy (EGD) which enables the physician to look inside the esophagus, stomach, and duodenum.

Although a number of illustrative variations are described above, it will be apparent to those skilled in the art that various changes and modifications may be made thereto without departing from the scope of the invention. Moreover, although specific configurations and applications may be shown, it is intended that the various features may be utilized in various types of procedures in various combinations as practicable. It is intended in the appended claims to cover all such changes and modifications that fall within the true spirit and scope of the invention.

What is claimed is:

1. A treatment system for engagement with a removable end effector, comprising an elongate flexible shaft adapted for endoluminal advancement within a patient and having a distal end adapted to engage a removable body portion of an end effector tool.

2. The system of claim 1 further comprising an end effector tool having a proximal end adapted to removably engage the distal end of the flexible shaft.

3. The system of claim 2 wherein the end effector tool is selected from the group consisting of graspers, forceps, scissors, snares, puncture needles, and needle knives.

4. The system of claim 2 wherein the end effector tool is energizable via RF, microwave, ultrasound, laser, or thermal energy.

5. The system of claim 1 wherein the distal end of the flexible shaft defines a channel for receiving and engaging a portion of the end effector tool.

6. The system of claim 1 further comprising an endoscopic device defining a lumen therethrough, wherein the flexible shaft is advanceable through the lumen.

7. The system of claim 6 wherein a profile of the end effector tool is larger than a diameter of the lumen.

8. The system of claim 6 wherein the endoscopic device is adapted to become rigidized along its length.

9. The system of claim 1 further comprising a handle assembly connected to a proximal end of the flexible shaft for controlling the end effector tool.

10. A treatment system for engagement with a removable end effector, comprising:
a flexible shaft adapted for endoluminal advancement within a patient and having a portion which is articulatable from a proximal end of the shaft, and
wherein a distal end of the shaft is adapted to engage a removable body portion of an end effector tool.

11. The system of claim 10 further comprising an end effector tool having a proximal end adapted to removably engage the distal end of the flexible shaft.

12. The system of claim 11 wherein the end effector tool is selected from the group consisting of graspers, forceps, scissors, snares, puncture needles, and needle knives.

13. The system of claim 11 wherein the end effector tool is energizable via RF, microwave, ultrasound, laser, or thermal energy.

14. The system of claim 10 wherein the distal end of the shaft flexible shaft defines a channel for receiving and engaging a portion of the end effector tool.

15. The system of claim 10 further comprising a handle assembly connected to a proximal end of the flexible shaft for controlling the end effector tool.

16. A treatment system for engagement with a removable end effector, comprising:
an elongate flexible shaft adapted for endoluminal advancement within a patient and having a distal end adapted to engage a removable body portion of an end effector tool; and
an endoscopic device defining a lumen therethrough, wherein the flexible shaft is advanceable through the lumen.

17. The system of claim 16 further comprising an end effector tool having a proximal end adapted to removably engage the distal end of the flexible shaft.

18. The system of claim 17 wherein the end effector tool is selected from the group consisting of graspers, forceps, scissors, snares, puncture needles, and needle knives.

19. The system of claim 17 wherein the end effector tool is energizable via RF, microwave, ultrasound, laser, or thermal energy.

20. The system of claim 16 wherein the endoscopic device is adapted to become rigidized along its length.

21. The system of claim 16 further comprising a visualization element positioned at a distal end of the endoscopic device.
